(19) United States
(12) Patent Application Publication
(10) Pub. No.: US 2013/0281405 A1
(43) Pub. Date: Oct. 24, 2013

(54) USE OF PC-NSAIDS TO TREAT AND/OR PREVENT PULMONARY INFLAMMATION

(71) Applicants: Lenard M. Lichtenberger, Houston, TX (US); Elizabeth J. Dial, Houston, TX (US)

(72) Inventors: Lenard M. Lichtenberger, Houston, TX (US); Elizabeth J. Dial, Houston, TX (US)

(21) Appl. No.: 13/855,624

(22) Filed: Apr. 2, 2013

Related U.S. Application Data
(60) Provisional application No. 61/619,265, filed on Apr. 2, 2012.

(51) Int. Cl. A61K 31/661

(52) U.S. Cl. CPC A61K 31/661
USPC 514/77

(57) ABSTRACT

Aerosolizable compositions of nonsteroidal anti-inflammatory drugs (NSAIDs) in combination with zwitterionic phospholipids in an aqueous carrier are used for treating lung injury (LI), pulmonary inflammation, and/or airway hyper-responsiveness (AHR). Methods for administering the compositions including orally, parenterally, intra-tracheally, and intra-pulmonarily protocols, especially intra-tracheally and intra-pulmonarily protocols, where the PC-NSAID compositions reduce lung injury (LI), pulmonary inflammation, and/or airway hyper-responsiveness (AHR).

### RELATED APPLICATIONS

This application claims the benefit of and priority to U.S. Provisional Patent Application Ser. No. 61/619,265, filed Apr. 2, 2012, the entire content of which is hereby incorporated by reference for all purposes as if set forth herein.

### BACKGROUND OF THE INVENTION

1. Field of the Invention

Embodiments of this invention relate to compositions for treating lung injury (LI), pulmonary inflammation and/or airway hyper-responsiveness (AHR) and methods for making and administering the compositions.

More particularly, embodiments of this invention relate to compositions for treating lung injury (LI), pulmonary inflammation and/or airway hyper-responsiveness (AHR) and methods for making and administering the novel compositions, where compositions include a nonsteroidal, anti-inflammatory drug (NSAID) or mixture of NSAIDs in combination with a zwitterionic phospholipid, a mixture of zwitterionic phospholipids or a phospholipid and an agent used for surfactant replacement therapy to form a PC-NSAID composition and the methods includes administering the composition parenterally and/or through the respiratory tract including intra-tracheally via inhalation or insufflation, where the PC-NSAID compositions reduce pulmonary inflammation and airway hyper-responsiveness (AHR). One embodiment of the present invention pertains to a composition properly diluted with an aqueous carrier, such as a saline or phosphate solution, so that the final composition can be atomized, made sprayable, or aerosolized, as a mist for inhalation.

2. Description of the Related Art

Lung disease is the third leading cause of death in the United States (U.S.), and more than 35 million Americans are currently afflicted with some form of acute or chronic lung disease (American Lung Association website). Pulmonary inflammation is a common feature of acute (i.e., ventilator-induced lung injury and acute respiratory distress syndrome (ARDS)) and chronic lung diseases (i.e., asthma chronic obstructive pulmonary disease (COPD), cystic fibrosis, and pneumonia). Furthermore, the degree of pulmonary inflammation often correlates with the severity of decrements in lung function (i.e., FEV₁, lung compliance, etc.). The net result of pulmonary inflammation and its subsequent effect on lung function is to reduce the efficiency of gas exchange, which becomes life-threatening, if uncorrected.

The treatment options for lung inflammation, and on a more chronic basis, COPD are limited to the use of inhaled corticosteroids and bronchodilators, antibiotics to treat primary and secondary microbial infections, and oxygen therapy. Although steroids can effectively treat inflammation, they are often associated with multiple, and serious side effects, including immunosuppression, anti-anabolic/catabolic actions on the musculoskeletal system, and their contributions to the development of imbalances in electrolytes and water in the various tissue compartments. These side-effects, notably the increased susceptibility to respiratory infection, and loss of respiratory muscle tone place the already compromised patient at further risk of developing irreversible pulmonary failure and death.

Surfactant replacement therapy by endotracheal administration of natural or synthetic surfactants extracted from porcine or bovine lung has been used pre-clinically with some success to treat LPS-induced acute lung inflammation/injury. However surfactant replacement therapy has not been translated into the clinic for treating lung injury and inflammation in older children and adults. Indeed, multiple clinical trials evaluating the efficacy of contrasting natural and synthetic surfactant formulations in the treatment of ARDS and related conditions associated with acute lung injury resulted in conflicting and equivocal results. One of the potential short-comings on the use of surfactant replacement therapy to treat LI and ARDS may relate to these conditions being clearly linked to pulmonary inflammation, providing a rationale for a combinatory approach with an anti-inflammatory agent, such as NSAIDs.

At present, nearly 120 million residents of the United States (U.S.) live in areas with ambient O₃ concentrations, which exceed the U.S. Environmental Protection Agency's National Ambient Air Quality Standards. Healthy individuals, who inhale O₃, exhibit pulmonary vascular hyperpermeability and pulmonary inflammation, cough and substernal soreness, decrements in pulmonary function, and airway hyper-responsiveness (AHR). O₃ also exacerbates respiratory symptoms in individuals with pre-existing lung disease, including asthma, COPD, and cystic fibrosis. Furthermore, increases in ambient O₃ concentrations are associated with short-term mortality. With air pollution continuing to be a persistent problem in the U.S. and with the large number of U.S. residents living in areas with unhealthy concentrations of O₃, exposure to O₃ and its associated detrimental health effects are significant public health concerns. Consequently, it is important to understand the mechanistic basis by which the respiratory system responds to O₃.

LPS is a pro-inflammatory glycolipid component of the cell wall of gram negative bacteria, which are present in inhaled air. Under normal conditions when LPS levels in the lung are modest, the body has effective defense mechanisms to combat this inciter of inflammation. In contrast, under conditions where the intra-pulmonary levels of LPS are high or the host's defense mechanism is compromised, an acute inflammatory response ensues which can be manifest at both the local (pulmonary) and systemic level. This LPS response is mediated by Toll-like receptor (TLR)-4, resulting in an increase in concentration of cytokine/chemokines in the BALF, neutrophil infiltration into the lung and an increased resistance to airflow. The LPS mouse model of acute pulmonary inflammation described by Haegens et al. (Haegens, A., P. Heeringa, R. J. van Suylen, C. Steele, Y. Aratani, R. J. O'Donoghue, S. E. Mutsaers, B. T. Mossman, F. F. Wouters, and J. H. Vernooy. 2009. Myeloperoxidase deficiency attenuates lipopolysaccharide-induced acute lung inflammation and subsequent cytokine and chemokine production. J Immunol 182:7990-7996) will be used to simulate this condition in our laboratory to evaluate the therapeutic efficacy of PC-NSAIDs to attenuate this process.

Although NSAIDs are the drug of choice for treating both acute inflammation, pain and fever and an expanding range of chronic inflammatory diseases, notably osteoarthritis. cardiovascular disease (thrombosis, stroke and angina), diverse neurological diseases (sciatica, Alzheimer's, Parkinson's) and cancer, NSAIDs are generally not used therapeutically for inflammatory lung diseases. This is likely a result of their contraindication in a small number of asthmatics, who have a tendency to bronchoconstrict following aspirin administration. However the underlying mechanism of aspirin-induced asthma is not clear, as prostaglandins which are generated by cyclooxygenase (COX) and generally induce smooth muscle relaxation have been linked to the development of bronchospasms associated with exercise. Interestingly, indomethacin, a well-known non-selective COX inhibitor, has been evaluated in the treatment of this condition with equivocal results. NSAIDs are rarely used to treat pulmonary inflammation, however one group has reported the effective use of orally administered high dose ibuprofen to alleviate pulmonary inflammation and improve lung function in cystic fibrosis patients. Thus, it is conceivable that PC-NSAIDs, especially if administered directly to the lung may prove to be both safe and effective for the treatment of patients suffering from acute and chronic inflammatory lung disease, that have both a history of being tolerant to aspirin without evidence of having an allergic response to it or other NSAIDs.

The therapeutic mechanism of action of indomethacin and ibuprofen appears to be primarily via inhibition of cyclo-oxygenase (COX), the enzyme responsible for the biosynthesis of prostaglandins and certain related eicosanoids. There are two COX isoforms, COX-1 and COX-2. COX-1 is a constitutive isoform found in platelets, GI mucosa and renal epithelia, while COX-2 is present in vascular endothelial cells and induced in settings of inflammation by cytokines and inflammatory mediators. NSAIDs, by and large, are organic acids that serve as reversible, competitive inhibitors of COX activity. Non-selective NSAIDs (i.e., those that inhibit both COX isoforms), not only diminish inflammation, but are ulcerogenic; this propensity for gastric or intestinal ulceration is partially due to the inhibition of GI mucosal COX, depleting the levels of cytoprotective prostaglandins. COX-2 selective inhibitors, also referred to as coxibs (i.e., those that selectively inhibit the COX-2 isoform), cause significantly less gastrointestinal damage, while achieving anti-inflammatory and analgesic efficacy. A family of coxibs, including rofecoxib, CELECOXIB, and VALDECOXIB were previously approved as GI-safer NSAIDs. However the coxibs all showed significantly increased risk of causing serious cardiovascular side effects, and all but CELECOXIB (which is a less selective COX-2 inhibitor) have been withdrawn from the market in the U.S.

In addition to inhibiting COX. NSAIDs have the capacity to chemically associate with phospholipids, notably phosphatidylcholine (PC) which are essential components of both cell membranes and extracellular barriers, that protect the gastrointestinal (GI) mucosal lining from luminal damaging agent (e.g., gastric HCl). This PC-NSAID interaction may in fact explain the surface damaging action of NSAIDs on the GI mucosa, resulting in both an attenuation in mucosal surface hydrophobicity and a decrease in the integrity of enterocyte membranes. Furthermore, it was demonstrated that this process together with the surface damaging action of NSAIDs could be significantly reduced or prevented if these drugs were pre-associated with either synthetic or purified PC prior to administration. Evidence that such a chemical interaction between PC and NSAIDs does occur include the findings that PC induces alterations in the solubility, melting point, and infrared spectroscopic characteristic of an NSAID.

Thus, there is a clear need in the art for compositions and methods for administering compositions to the pulmonary system to reduce pulmonary inflammation.

### SUMMARY OF THE INVENTION

Embodiments of the present invention provide compositions including at least one nonsteroidal anti-inflammatory drug (NSAID) and at least one zwitterionic phospholipid or at least one phospholipid and at least one lung replacement surfactant composition to form PC-NSAID compositions, where the PC-NSAID compositions reduce lung injury (LI), pulmonary inflammation, and/or airway hyper-responsiveness (AHR). One embodiment of the present invention pertains to a composition that can be atomized, made sprayable as an aerosol for inhalation. The desired composition is properly diluted with a carrier such as an aqueous saline solution, water, or a phosphate buffer.

Embodiments of the present invention provide methods for treating pulmonary inflammation and airway hyper-responsiveness (AHR) comprising administering a compositions including at least one nonsteroidal anti-inflammatory drug (NSAID) and at least one zwitterionic phospholipid or at least one phospholipid and at least one lung replacement surfactant composition to form PC-NSAID compositions, parenterally and through the respiratory tract including intratracheally, by inhalation and by insufflation to a patient, where the PC-NSAID compositions reduce lung injury (LI), pulmonary inflammation, and/or airway hyper-responsiveness (AHR).

Embodiments of the present invention provide methods for treating pulmonary inflammation and airway hyper-responsiveness (AHR) comprising administering a compositions including at least one nonsteroidal anti-inflammatory drug (NSAID) and at least one zwitterionic phospholipid or at least one phospholipid and at least one lung replacement surfactant to form PC-NSAID compositions, intra-tracheally, and/or intra-pulmonarily to a patient, where the PC-NSAID compositions reduce lung injury (LI), pulmonary inflammation, and/or airway hyper-responsiveness (AHR).

Embodiments of this invention relate to compositions for reducing lung injury (LI), pulmonary inflammation, and/or airway hyper-responsiveness (AHR) including at least one nonsteroidal anti-inflammatory drug (NSAID) and at least one zwitterionic phospholipid, where the composition reduces lung injury (LI), pulmonary inflammation, and/or airway hyper-responsiveness (AHR). In certain embodiments, the compositions may also include at least one lung replacement surfactant composition. In other embodiments, the phospholipids are pre-associated with the NSAID. In other embodiments, the phospholipids are pre-associated with the NSAID and the lung replacement surfactant compositions are formulated with the NSAID. In other embodiments, the phospholipids are selected from the group of phosphatidylcholine class of phospholipids. In other embodiments, the phospholipids are selected from the group of phosphatidylcholines such as phosphatidyl choline (PC), dipalmitoylphosphatidylcholine (DPPC), other disaturated phosphatidylcholines, or mixtures and combinations thereof. In other embodiments, the lung replacement surfactant composition are selected from the group consisting of porcine lung extracts, bovine lung extracts, synthetic analogs, and mixtures or combinations thereof. In other embodiments, the composition further include water or an aqueous carrier to form a diluted composition.

Embodiments of this invention relate to methods for reducing lung injury (LI), pulmonary inflammation, and/or airway hyper-responsiveness (AHR) including administering a composition comprising at least one nonsteroidal anti-inflammatory drug (NSAID) and at least one zwitterionic phospholipid, where the composition reduces lung injury (LI), pulmonary inflammation, and/or airway hyper-responsiveness (AHR). In certain embodiments, the methods further include prior to the administering step, diluting the composition with water or an aqueous carrier to form a diluted composition. In other embodiments, the administering step includes parenterally administering the composition. In other embodiments, the administering step includes intratracheally administering the composition. In other embodiments, the intratracheally administering step includes intratracheally administering via inhalation. In other embodiments, the intratracheally administering step includes intratracheally administering insufflation. In other embodiments, the intratracheally administering step includes spraying a mist of the composition into the pulmonary system by inhalation through the throat or nose. In other embodiments, the methods further include the step of producing the mist by atomizing the composition. In other embodiments, the methods further include producing the mist by nebulizing or aerosolizing the composition. In other embodiments, the administering step includes parenterally administering the diluted composition. In other embodiments, the administering step includes intratracheally administering the diluted composition. In other embodiments, the intratracheally administering step includes intratracheally administering via inhalation. In other embodiments, the intratracheally administering step includes intratracheally administering insufflation. In other embodiments, the intratracheally administering step includes spraying a mist of the composition into the pulmonary system by inhalation through the throat or nose. In other embodiments, the methods further include the step of producing the mist by atomizing the diluted composition. In other embodiments, the methods further include the step of producing the mist by nebulizing the diluted composition. In other embodiments, the mist is formed using air, oxygen, or a nitrogen and oxygen mixture.

### BRIEF DESCRIPTION OF THE DRAWINGS

The invention can be better understood with reference to the following detailed description together with the appended illustrative drawings in which like elements are numbered the same:

FIG. 1 depicts two views of a computer generated structure of a possible PC:Ibuprofen complex determined by r-MD calculations based on direct ¹H-¹H interactions observed in a 300 ms ROESY NMR experiment.

FIG. 2 depict results (n=3-5 mice/group) demonstrating the anti-inflammatory activity of parenterally administered Indomethacin (Indo) vs PC-Indo to inhibit ozone (Oz)-induced pulmonary inflammation-based upon cell counts (bars) and protein concentration (line) of BALF, 24 hr post-O₃ exposure.

FIG. 3 depicts the effects of O₂ exposure on cell counts in BALF, indicating that O₃ increased white cell count vs. room air and endotracheal administration of PC:Indomethacin significantly reduced the white cell counts in the BALF, both in mice exposed to the pollutant and those that are exposed to room air. No decrease in cell count was observed with the NSAID or PC alone, in fact the cell count was increased.

FIG. 4 depicts the effects of O₃ exposure on protein concentration of the BALF, indicating that O₃ increased shedding of protein into the lung fluid vs room air and endotracheal administration of PC-Indomethacin significantly reduced the BALF protein conc, both in mice exposed to the pollutant and those that are exposed to room air. No such effect was observed with the NSAID or PC alone

FIG. 5 depicts the effects of O₃ exposure on myeloperoxidase (MPO) activity in BALF, indicating that O₃ increased the activity of this neutrophil enzyme vs room air and endotracheal administration of PC-Indomethacin significantly reduced the BALF MPO activity, both in mice exposed to the pollutant and those that are exposed to room air. No such effect was observed with the NSAID or PC alone

FIG. 6 depicts the effects of O₃ exposure on PGE₂ concentration of the BALF, indicating the generation of the inflammatory eicosanoid into the lung fluid occurred in mice exposed to O₃ and room air and that both PC-Indomethacin and the NSAID alone appeared to have efficacy to significantly inhibit PGE₂ conc of the collected lung fluid.

FIG. 7 depicts the effects of O₃ exposure on protein concentration of the BALF, indicating that O₃ increased shedding of protein into the lung fluid vs room air (vehicle w/o O₃) and endotracheal administration of another PC-NSAID namely PC-Ibuprofen, significantly reduced the BALF protein conc of O₃-challenged mice when administered by endotracheal tube at doses of 2, 5 and 10 mg/kg.

FIG. 8 depicts the stability of Indomethacin (Indo) and PC-Indomethacin (PC-Indo) before and after sterile filtration, when reconstituted in PBS or sodium bicarbonate buffers and stored at 4° C.

FIGS. 9A-D depict evidence that PC-Ibuprofen (PC-IBU) (PC is DPPC) has efficacy to reduce ozone (Oz)—induced lung injury/inflammation as indicated by an attenuation of BALF; (A) leukocytes; (B) protein; (C) MPO activity; and (D) PGE2.

FIG. 10 depicts an aerosol test system used in this invention.

FIG. 11 depicts the Particle Size Distribution for all runs at various PBS dilution factors for the PC-Ibuprofen complex (PC is LIPOID S100). Note that FIG. 11 shows nebulizer output averaged for the entire 3 minute run.

FIG. 12 depicts the cumulative mass concentration versus particle size diameter for various PBS dilutions of the PC-Ibuprofen complex (PC is LIPOID S100). From this graph we can see for most runs that a majority of the mass (˜80%) falls in the region below 6 μm.

FIG. 13 depicts the estimated patient delivery rates for PC:Ibuprofen complex (PC is LIPOID S100) with different PBS dilution ratios. From this graph, patient treatment time may be estimated based on a desired mass of ibuprofen delivered to the patient.

### DETAILED DESCRIPTION OF THE INVENTION

The inventors have found that compositions may be formulated for treating lung injury (LI), pulmonary inflammation, and/or airway hyper-responsiveness (AHR), where the compositions include zwitterionic phospholipid-NSAID non-covalent association complexes or zwitterionic phospholipid-NSAID non-covalent association complexes and lung surfactant replacement composition complexes with NSAID. The phospholidips are preferably from the phosphatidylcholine class of phospholipids and constitute a major component of cellular membranes and pulmonary surfactants and other biological surface barriers layers. We have evaluated the therapeutic efficacy/potency of PC-NSAIDs on reducing pulmonary inflammation and AHR in response to ozone (O₃), and we believe PC-NSAIDs would be effective treatments for LPS induced pulmonary damage and/or smoke inhalation from fires, tobacco, or marijuana. O₃ is a highly reactive, oxidant gas and the major component of photochemical smog. To our knowledge. PC-NSAID or PC-NSAID and lung surfactant replacement composition technology has not been applied to the treatment of pulmonary inflammation, nor the effects of administering PC-NSAID formulations on lung function after pulmonary administration been studied.

Embodiments of the present invention relate broadly to compositions including at least one nonsteroidal anti-inflammatory drug (NSAID) and at least one zwitterionic phospholipid or at least one phospholipid and at least one lung replacement surfactant composition to form PC-NSAID compositions, where the PC-NSAID compositions reduce lung injury (LI), pulmonary inflammation, and/or airway hyper-responsiveness (AHR). Embodiments of the present inventions that contain from about 1:1 to about 5:1 molar ratios of the phospholipid to NSAID.

Embodiments of the present invention relate broadly to methods for treating pulmonary inflammation and airway hyper-responsiveness (AHR) comprising administering a compositions including at least one nonsteroidal anti-inflammatory drug (NSAID) and at least one zwitterionic phospholipid or at least one phospholipid and at least one lung replacement surfactant composition to form PC-NSAID compositions, parenterally and through the respiratory tract including intratracheally, by inhalation and by insufflation to a patient, where the PC-NSAID compositions reduce lung injury (LI), pulmonary inflammation, and/or airway hyper-responsiveness (AHR). The intratracheal or intrapulmonary administration may include spraying a mist of the compositions into the pulmonary system by inhalation through the throat or nose. The mist may be produced by pumping the composition through an orifice and entraining the composition into an air stream, where the air may be supply from a compressed air source. Another method includes vaporizing the compositions of this invention to form a vapor with aerosolized or nebulized PC-NSAID droplets for inhalation through the throat or nose into the pulmonary system. Yet another method may include heating the composition in the present of a warm vapor stream, where the warm vapor stream may be warm air, warm moist air, or warm water vapor. Still another method includes forming a mist including a composition of this invention using water as the misting agent, where the water is pumped into a stream of the composition through an orifice that results in the formation of a mist of the composition and water. The mists may be formed using standard nebulizers, atomizers, continuous positive airway pressure (CPAP) devices, and/or CPAP humidifier technology, especially nebulizers or atomizers having single orifices or concentric orifices for introducing the composition, a secondary carrier or agent and a gas to produce the mist for inhalation. Besides water, the above technology may use any bio-compatible aqueous carrier. The gas may be air, oxygen, or an oxygen and nitrogen gas mixture.

Embodiments of the present invention relate broadly to methods for treating pulmonary inflammation, lung injury, and airway hyper-responsiveness (AHR) comprising administering a compositions including at least one nonsteroidal anti-inflammatory drug (NSAID) and at least one zwitterionic phospholipid or at least one phospholipid and at least one lung replacement surfactant to form PC-NSAID compositions, intra-tracheally, and/or intra-pulmonarily to a patient, where the PC-NSAID compositions reduce lung injury (LI), pulmonary inflammation, and, or airway hyper-responsiveness (AHR).

We have evaluated the potential therapeutic efficacy and potency of two PC-NSAIDs (PC-Ibuprofen and PC-Indomethacin) vs unmodified ibuprofen and indomethacin, respectively, administered by contrasting routes of administration (parenteral, intratracheal, and/or intrapulmonary) to attenuate pulmonary inflammation in rodent model systems of pulmonary inflammation. The animal model we have studied to date is one of acute lung injury where mice are treated with our formulations before and after they are exposed to the toxic environmental agent ozone (O₃), which reproducibly induces lung injury (LI) and pulmonary inflammation in this animal species. Pulmonary inflammation has (in results presented below) been measured by assessing release of neutrophils and its marker enzyme, myeloperoxidase (MPO), proteins, and pro-inflammatory prostaglandins (PGE2) and cytokines released into the animal's bronchoalveolar lavage fluid (BALF).

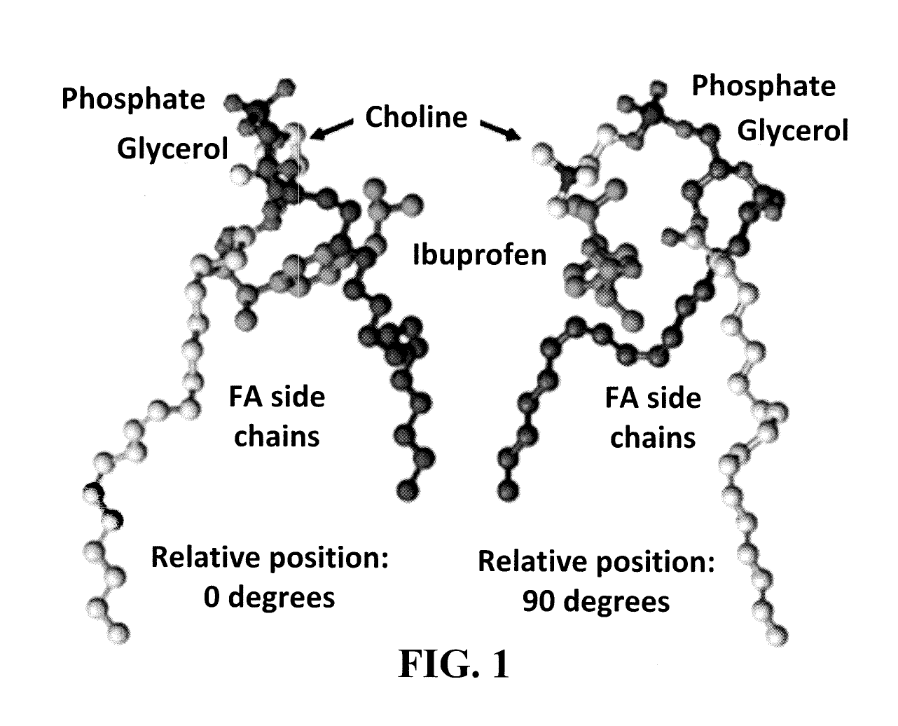

A recently performed NMR analysis revealed that ibuprofen and PC form both ionic and hydrophobic atomic associations that are non-covalent in nature, based upon the amphipathic properties of the two classes of molecules as shown in FIG. 1.

There are a number of significant innovations in this invention. First is the use of NSAIDs to treat pulmonary inflammation and related diseases that develop neutrophilic pulmonary inflammation. The use of NSAIDs for such treatments would not be obvious to an ordinary artisan due to concerns that patients may have an allergic reaction to aspirin and related NSAIDs resulting in bronchoconstriction. However, such patients make up only a small subset of asthmatics (5-10%) relative to the 35 million Americans suffering from chronic lung diseases. Furthermore PC or various types of surfactant replacement therapy have not proven to be effective in treating pulmonary inflammation or lung injury on its own in older children (other than preterm neonates) or adults. The second innovation therefore, is the use of composition including PC-NSAID complexes to treat pulmonary inflammation, LI and/or AHR.

To facilitate the intrapulmonary administration of PC-Ibuprofen and PC-Indomethacin based compositions, we have modified a literature method to deliver a drug to an anesthetized mouse by endotrachel intubation.

To facilitate the intra-pulmonary administration of Ibuprofen-PC and Indomethacin-PC based compositions, we have modified a literature method to deliver a drug via inhalation or aerosolation.

### Reagents Suitable for Use in the Invention

Suitable biocompatible, zwitterionic phospholipids for use in this invention include, without limitation, a phospholipid of general formula:

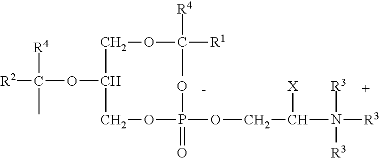

where R¹ and R² are saturated or unsaturated substitutions ranging from 8 to 32 carbon atoms; R³ is H or CH₃, and X is H or COOH; and R⁴ is ═O or H₂. Mixtures and combinations of the zwitterionic phospholipids of the general formula and mixtures and combinations of NSAIDs can be used as well.

Exemplary examples of zwitterionic phospholipid of the above formula include, without limitation, phosphatidylcholines such as phosphatidyl choline (PC), dipalmitoylphosphatidylcholine (DPPC), other disaturated phosphatidylcholines, phosphatidylethanolamines, phosphatidylinositol, phosphatidylserines sphingomyelin or other ceramides, or various other zwitterionic phospholipids, phospholipid containing oils such as lecithin oils derived from soy beans, dimvristoylphosphatidylcholine, distearoylphosphatidylcholine, dilinoleoylphosphatidylcholine (DLL-PC). dipalmitoylphosphatidylcholine (DPPC), soy phophatidylchloine (Soy-PC or PC_{S}) and egg phosphatidycholine (Egg-PC or PC_{E}). In DPPC, a saturated phospholipid, the saturated aliphatic substitution R₁ and R₂ are CH₃—(CH₂)₁₄, R₃ is CH₃ and X is H. In DLL-PC, an unsaturated phospholipid, R₁ and R₂ are CH₃—(CH₂)₄—CH══CH₂CH₂—CH══CH—(CH₂)₇, R₃ is CH₃ and X is H. In Egg PC, which is a mixture of unsaturated phospholipids, R₁ primarily contains a saturated aliphatic substitution (e.g., palmitic or stearic acid), and R₂ is primarily an unsaturated aliphatic substitution (e.g., oleic or arachidonic acid). In Soy-PC, which in addition to the saturated phospholipids (palmitic acid and stearic acid) is a mixture of unsaturated phospholipids (oleic acid, linoleic acid and linolenic acid). In certain embodiments, the phospholipids are zwitterionic phospholipid include, without limitation, dipalmitoyl phosphatidylcholine, phosphatidyl choline, or a mixture thereof.

Suitable NSAIDS include, without limitation: (a) propionic acid drugs including fenoprofen calcium, flurbiprofen, suprofen, benoxaprofen, ibuprofen, ketoprofen, naproxen, and/or oxaprozin; (b) acetic acid drug including diclofenac sodium, diclofenac potassium, aceclofenac, etodolac, indomethacin, ketorolac tromethamine, and/or ketorolac; (c) ketone drugs including nabumetone, sulindac, and/or tolmetin sodium; (d) fenamate drugs including meclofenamate sodium, and/or mefenamic acid; (e) oxicam drugs piroxicam, lornoxicam and meloxicam; (f) salicylic acid drugs including diflunisal, aspirin, magnesium salicylate, bismuth subsalicylate, and/or other salicylate pharmaceutical agents; (g) pyrazolin acid drugs including oxyphenbutazone, and/or phenylbutazone; and (h) mixtures or combinations thereof.

Suitable COX-2 inhibitors include, without limitation, celecoxib, rofecoxib, or mixtures and combinations thereof.

Suitable surfactants for lung replacement therapy include, without limitation, natural pulmonary surfactants, synthetic pulmonary surfactants, and mixtures or combinations thereof. Natural pulmonary surfactants include, without limitation, porcine lung extract, bovine lung extract, and mixtures or combinations thereof. Naturally pulmonary surfactants contain about 40% dipalmitoylphosphatidylcholine (DPPC), about 40% other phospholipids (PC), about 5% surfactant-associated proteins (SP-A, B, C and D), cholesterol (neutral lipids) and traces of other substances. Exemplary examples of animal derived lung surfactants includes, without limitation. Alveofact®, a registered trademark of Lyomark Pharma GmbH of Oberhaching, Germany, extracted from cow lung lavage fluid, CUROSURF®, a registered trademark Cornerstone Therapeutics Inc., Cary, N.C., extracted from material derived from minced pig lung, INFASURF®, a registered trademark of ONY, Inc., Amherst, N.Y., (calfactant), extracted from calf lung lavage fluid, SURVANTA®, a registered trademark of Abbvie Inc. Corporation Delaware, (beractant), extracted from minced cow lung with additional DPPC, palmitic acid and tripalmitin, and mixtures or combinations thereof. Exemplary examples of synthetic pulmonary surfactants include, without limitation, EXOSURF™ available from Glaxo Wellcome, a mixture of DPPC with hexadecanol and tyloxapol added as spreading agents, pumactant, an artificial lung expanding compound, a mixture of DPPC and PG, KL-4, a lung surfactant material composed of DPPC, palmitoyl-oleoyl phosphatidylglycerol, and palmitic acid, combined with a 21 amino acid synthetic peptide that mimics the structural characteristics of SP-B, Venticute®, a registered trademark of NYCOMED GMBH CORPORATION FED REP GERMANY, composed of DPPC, PG, palmitic acid and recombinant SP-C, SURFAXIN®, a registered trademark of Acute Therapeutics, Inc., (lucinactant) composes of dipalmitoylphosphatidylcholine, 1-palmitoyl-2-oleoyl-sn-glycero-3-phosphoglycerol, and palmitic acid, Aerosurf™ (aerosolized form of SURFAXIN®), and mixtures or combinations thereof.

### EXPERIMENTS OF THE INVENTION

The basic design of the experiments of this invention is first to evaluate the effects of lung injury (LI) in mice exposed to a chemical agent (ozone) or a biological (LPS) agent on measures of pulmonary inflammation. Pilot studies were performed to evaluate the anti-inflammatory efficacy and safety of ibuprofen or as a purified complex with (soy)/synthetic (dipalmitoyl) PC. In these experiments, we also compared the anti-inflammatory activity of PC-Ibuprofen vs unmodified Ibuprofen. All of the above surfactant test agents with the appropriate controls (PBS and NSAID alone) were dosed by an intrapulmonary route of administration and compared to parenteral administration routes, after challenge with these injurious agents. Pulmonary inflammation was assessed primarily by analyzing cytokines, chemokines, and inflammatory cells in BALF; pulmonary barrier properties were assessed by measuring BALF protein and albumin concentrations.

### Experiment 1

To evaluate the potential therapeutic efficacy and potency of two PC-NSAIDs (PC-Ibuprofen and PC-Indomethacin) compositions vs their respective unmodified NSAID (ibuprofen and indomethacin), the composition were administered by contrasting routes of administration (parenteral or intrapulmonary) to attenuate LI, pulmonary inflammation, and/or airway hyper-responsiveness (AHR) in a mouse model of ozone-induced pulmonary inflammation.

As outlined previously, ozone (O₃) is a highly reactive, oxidant gas, present in smog, that is known to cause pulmonary inflammation, a decrement in pulmonary function, a cough and development of airway hyperresponsiveness (AHR).

The concentration of O₃ (2 ppm) used in these studies exceeds the current U.S. Environmental Protection Agency standard of 0.08 ppm ozone. When mice were exposed to 2 ppm O₃, their minute ventilation decreases as much as two-thirds. The total dose of O₃ delivered to the lungs is the product of O₃ concentration, exposure time, and minute ventilation. On the other hand, humans are exposed to lower concentrations of O₃, but exercise increases their minute ventilation, and subsequently, the total dose of O₃ delivered to their lungs. Thus, although the O₃ concentrations mice and humans are exposed to under experimental conditions are quite different; the dose of O₃ inhaled into the lungs may be very similar. Therefore, if we observe a positive or beneficial effects from PC-NSAID formulation administration post O₃ exposure at high O₃ concentration, we believe that the PC-NSAID formulations should be efficacious in treating patients exposed to more environmentally relevant concentrations.

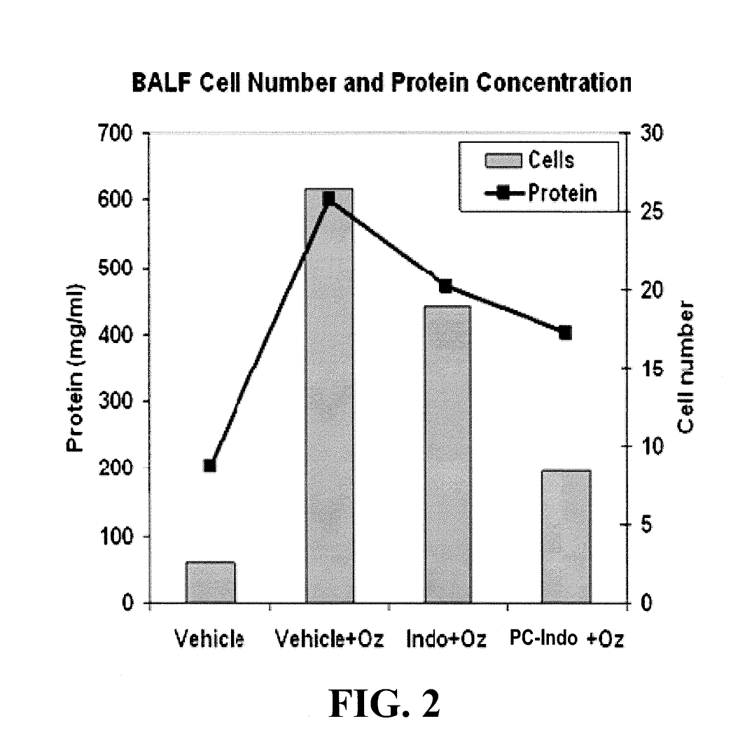

Using the O₃-induced pulmonary inflammation model, we have evaluated and compared the anti-inflammatory efficacy of PC-Indomethacin vs indomethacin administered parenterally at a dose of 10 mg/kg vs vehicle control and evaluated the BALF 24 h post exposure. The white cell counts and protein concentrations of the BALF of the test groups are depicted in FIG. 2. It can be appreciated that in this pilot study, both indomethacin and PC-Indomethacin appeared to inhibit this index of O₃-induced pulmonary inflammation, with the PC-NSAIDs trending to be more efficacious than the parent drug alone.

Based upon these encouraging findings where the PC-NSAID was administered parenterally to inhibit O₃-induced pulmonary inflammation, we initiated a series of experiments where the PC-Indomethacin composition and PC-Ibuprofen composition were administered directly to the lungs via endotracheal administration.

Because of the clinical relevance of environmental ozone exposure, and the experience of our team, with the murine model of O₃-induced LI, pulmonary inflammation, and/or AHR, we have evaluated the anti-inflammatory efficacy of our PC-NSAID formulations using this robust model system. In these studies, all mice were pre-dosed (via endotracheal administration) with vehicle, indomethacin (at a dose range from 2 mg/kg), ibuprofen (5 mg/kg) or the equivalent (NSAID) doses of the PC-NSAID compositions, 1 h before and 1.5 hours after being exposed to either filtered room air or O₃ (2 ppm) for 3 h and then 6 h or 24 h following the cessation of exposure airway pulmonary injury and inflammation were assessed by euthanizing the animals and collecting bronchoalveolar lavage fluid (BALF) using standard techniques. The O₃ exposure protocol were chosen to allow for comparison with data of other investigators studying acute O₃-induced pulmonary inflammation/AHR in mice. The levels of inflammatory mediators (IL-6, MIP-2, KC, myeloperoxidase activity/MPO) in the BALF at 6 h and 24 h following the cessation of O₃ exposure were determined because previous data studies indicated that these levels are highest between 4 h and 6 h post-exposure in wild-type mice. However, at 24 h post-exposure, airway responsiveness to MCh and the levels of BALF protein and the number of BALF neutrophils are at their highest in wild-type and obese mice.

In these studies, all mice were pre-dosed with vehicle (PBS), NSAID (indomethacin or ibuprofen) (at a dose of 2 mg NSAID/kg), or the equivalent (NSAID) dose of the corresponding PC-NSAID composition using an endo-tracheal delivery method refined that we refined (25 μL/rat) 1 h before and 90 minutes after being exposed to either filtered room air or O₃ (2 ppm) for 3 h. The PC-Indomethacin composition was prepared dissolving indomethacin and purified PC (Phospholipon 90G from Lipoid) in a polar solvent (such as acetone), followed by vacuum removal of the solvent to form a purified PC-NSAID oil composition. This purified PC-NSAID oil composition is then added to an amount of phosphate buffered saline (PBS) followed by 30 minutes of sonication to provide a uniform composition at the appropriate dose for intra-tracheal administration. In some experiments, we prepared the PC-Indomethacin composition using Lipoid S-100 (a highly purified >98% soy PC product, as recommended by the manufacturer) instead of Phospholipon 90G, as well as comparing the effects of the purified soy PC products (S-100 and 90G) alone. As the results with S-100 and 90G were not different, we opted to pool the results obtained from the two Lipoid purified soy PC products. Twenty-four hours (24 h) following the cessation of O₃ exposure, pulmonary injury and inflammation were assessed by euthanizing the animals and collecting bronchoalveolar lavage (BALE) fluid using standard techniques. The collected BALF samples were centrifuged (with the cell pellet analyzed for white cells) and the supernatant analyzed for protein and prostaglandin E2, all markers of pulmonary inflammation.

### Results

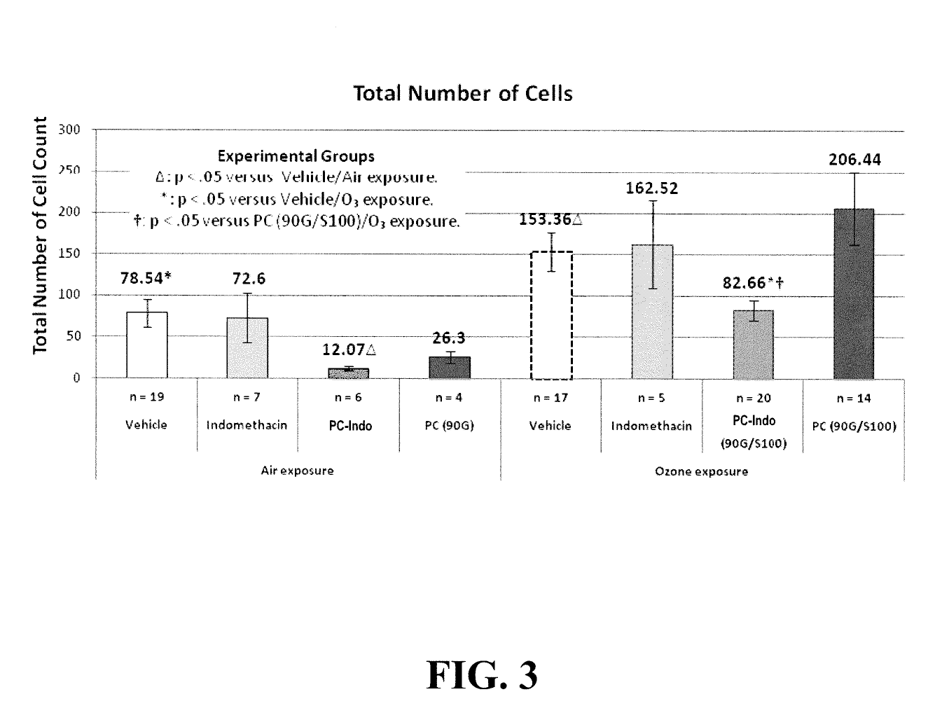

The pooled results of ˜9 experiments are summarized below. The data shown in FIG. 3 demonstrated that O₃ exposure significantly increased white cell count vs room air. The data also demonstrated that endotracheal administration of PC-Indomethacin significantly reduced the white cell count in the pulmonary lavage fluid, BALF, both in mice exposed to the pollutant and those that were exposed to room air. In contrast no such anti-inflammatory effect was observed in mice intra-tracheally administered the NSAID alone or PC alone, both of which showed increased white cell count relative to vehicle. It was also noted that treatment with the PC-NSAID and PC alone significantly reduced cell numbers in the pulmonary lavage fluid of mice exposed to room air (vs vehicle control values) when both test agents were administered by endotracheal tube.

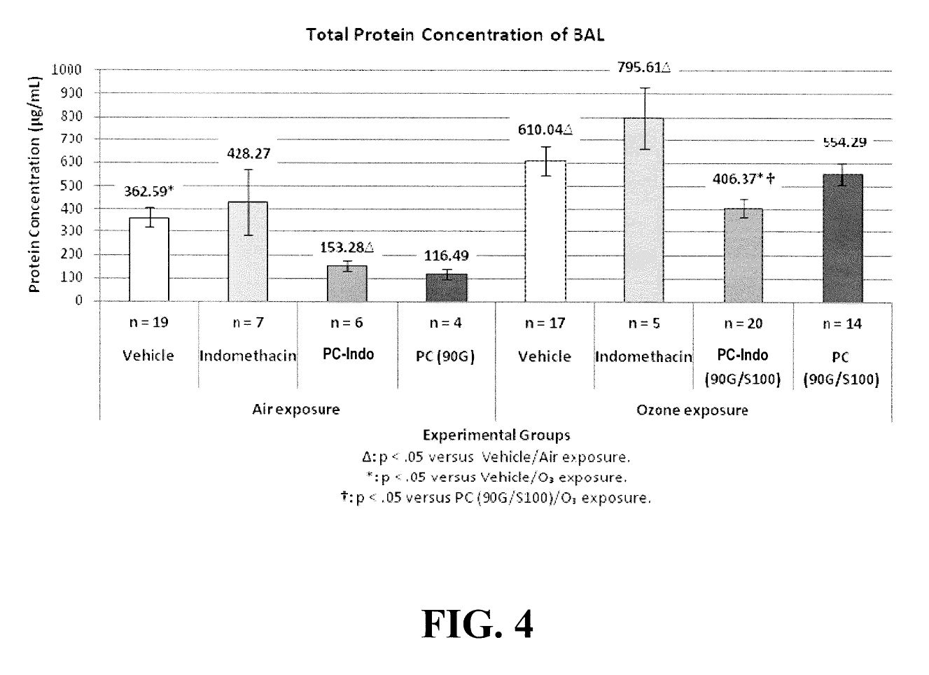

Referring now to FIG. 4, a similar effect was observed, when we measured the protein concentration of the pulmonary lavage fluid, with O₃ exposure inducing a statistically significant increase vs. room air, which were completely and significantly reversed in mice endotracheally administered PC-Indomethacin, whereas the unmodified NSAID and PC alone did not cause a similar effect. Interestingly, the PC-NSAID and PC alone also reduced the protein concentration in the lavage fluid of mice exposed to room air vs. those administered the PBS vehicle by endotracheal tube.

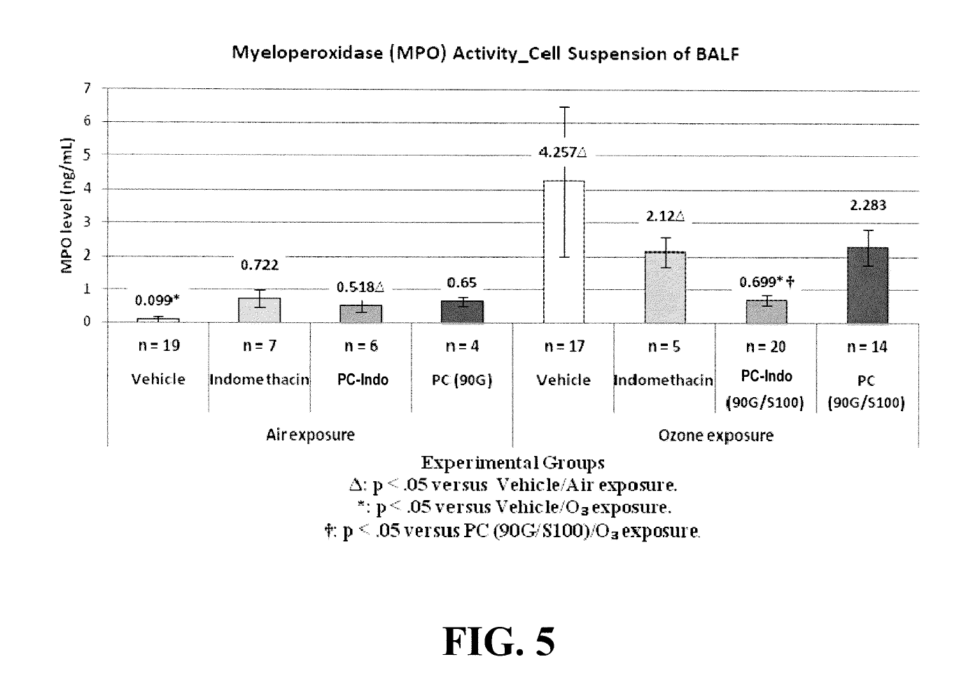

We also performed a biochemical assay to measure the neutrophilic enzyme myeloperoxidase (MPO) in the lavage fluid, and the results depicted in FIG. 5, demonstrate that a similar pattern was recorded as for BALF cell number, with ozone-inducing an increase in MPO activity which was significantly reduced by PC-Indomethacin, but not by the indomethacin or PC alone.

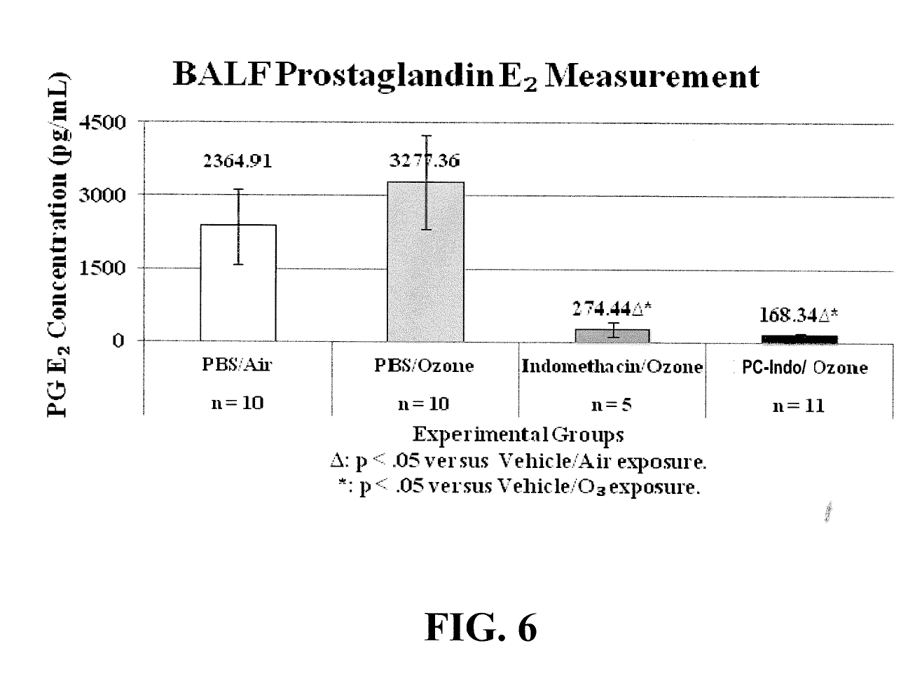

We also measured the inflammatory mediator, PGE₂ in the pulmonary lavage fluid, which showed a somewhat different pattern as shown in FIG. 6. Here it can be appreciated that the concentration of the pro-inflammatory eicosanoid was elevated in both mice exposed to room air and O₃ with the latter group being somewhat higher. If we focus on the mice exposed to the pollutant, it can also be appreciated that both PC-Indomethacin and the NSAID alone had a statistically significant effect to markedly inhibit PGE₂ generation, which appears to be mostly related to mechanical injury of the lung related to the method of administration of the test articles.

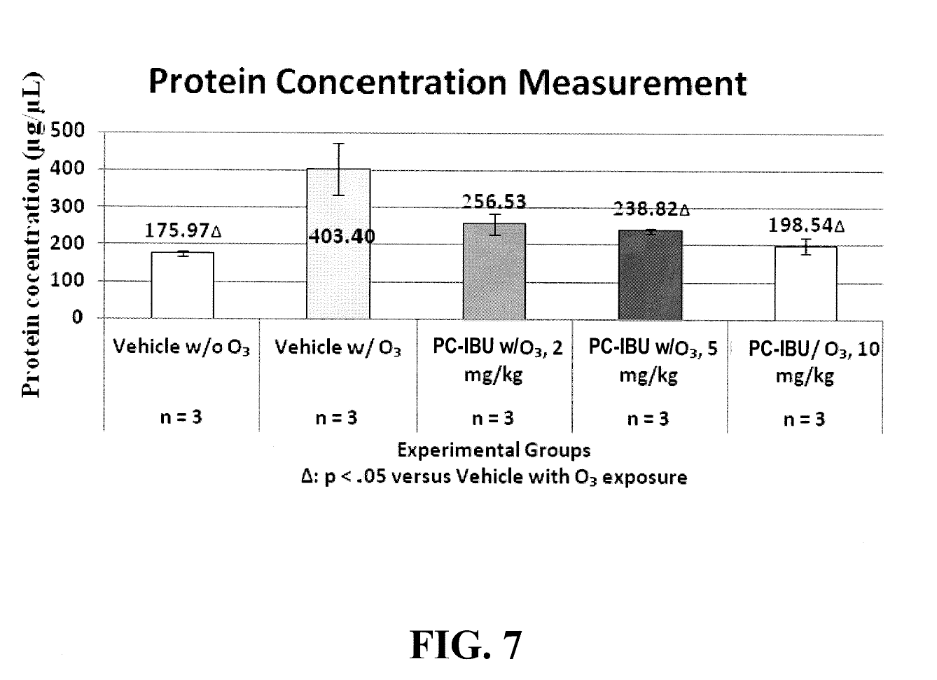

In a pilot study we investigated the effects of endotracheally administered PC-Ibuprofen to block O₃-induced pulmonary inflammation. The results of this dose-response study, where we limited our analysis to the measurement of protein concentration of the pulmonary lavage fluid are shown in FIG. 7. It can be appreciated that PC-Ibuprofen at all concentrations tested (tested at 2, 5 and 10 mg/kg, administered via endotrach tube) all significantly inhibited this marker of pulmonary inflammation vs the O₃-challenged control rats that were dosed with PBS by the same route of administration.

### Biochemical & Cellular Assays

The concentration of total BALF protein has been determined spectrophotometrically according to the Bradford protein assay procedure (Bio-Rad Laboratories, Inc.; Hercules, Calif.) while the levels of IL-6, MIP-2, PGE₂ in the BALF are determined using a commercially available ELISA kit (Immunology Consultants Laboratories, Inc., Newberg, Oreg.), and MPO activity (by enzymatic kit provided CytoStore) in accordance to the manufacturer's instructions. Pulmonary inflammation has been determined by assessing the BALF cell differentials on Cytospin cytocentrifuge preparations. Hemoglobin has been measured by the benzidine assay.

### Experiment 2

### Preparation of PC-NSAIDS for Parenteral and Intra-Pulmonary Delivery

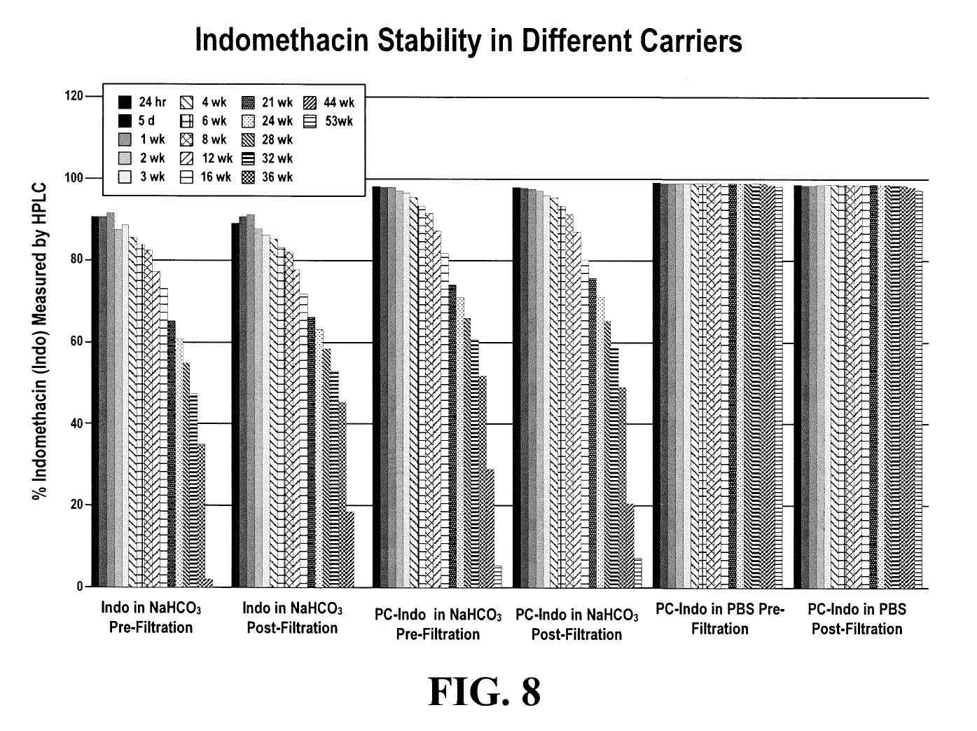

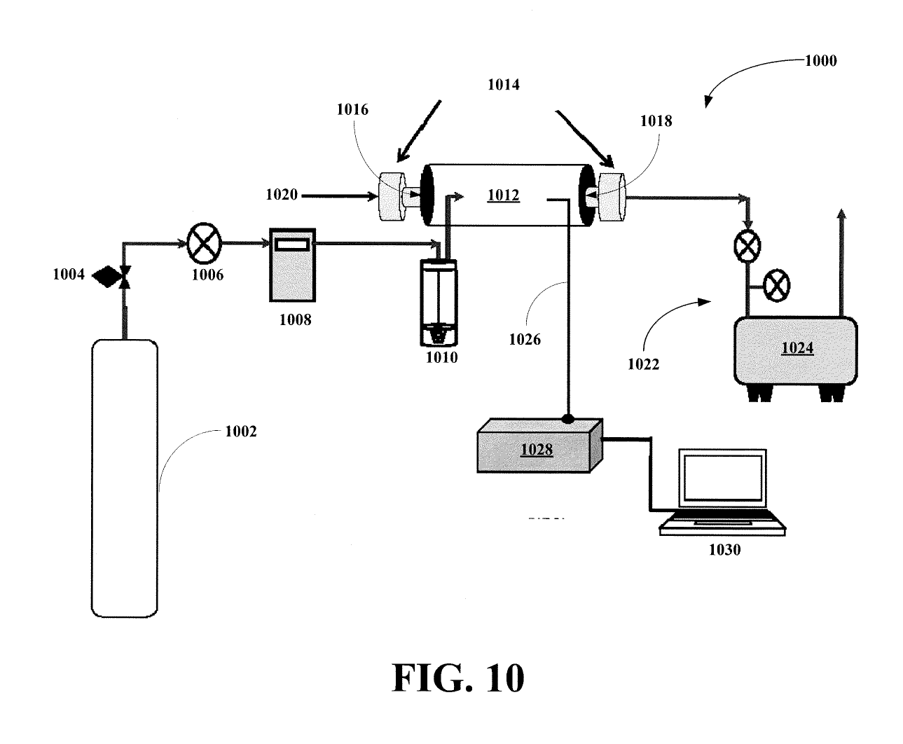

The proprietary method used to prepare the purified PC-NSAIDs takes advantage of the fact that pre-dissolving indomethacin (or ibuprofen) in acetone beforehand markedly increases the solubility of PC in this polar solvent (normally PC has a very limited solubility in acetone). Thereby the NSAID and purified soy PC (Lipoid S-100) (1:1 PC to NSAID molar ratio, which correspond to 2:1 PC to NSAID weight ratio) are dissolved in acetone, in this order and incubated at 40° C. until the solution clarifies. This solution is then placed in a rotor-evaporator for at least 12 hours to remove the volatile solvent. In the case of disaturated PC, such as dipalmitoyl-PC (DPPC) (which is the major phospholipid in pulmonary surfactant) we have learned that it is important to have 2-4 times more NSAID than PC (on a molar basis) to drive the solubility of the DPPC into the polar solvent. The resulting clear oil, which has been found to have high concentrations of the PC-NSAID complex, can be readily dispersed in phosphate buffered saline (PBS) or other biologically compatible solvents and sterilized by Millipore filtration. The PC-Indomethacin oil (or PC-Ibuprofen) and PBS sterile filtrate have been tested for stability when stored at under refrigeration, and both the NSAID and PC constituents have been found to be stable for up to one year. FIG. 8 demonstrates that PC-Indomethacin (called Indo:90G) prepared in PBS (either un-filtered or sterile filtered) and stored at 4° C. remains stable (last two sets of bars on right) as opposed to when the PC-NSAID or unmodified indomethacin is dispersed in sodium bicarbonate buffer, where it degrades over time. With the advice of the soy lecithin manufacturer (Lipoid Inc., Heidelberg, Germany) we are transitioning to their more purified soy lecithin product (Lipoid S-100) which already is approved for parenteral administration. Because use of this more purified soy lecithin product will also meet FDA stringent cGMP requirements for parenteral formulations, we propose to use Lipoid S-100 in all future parenteral and aerosol formulations. FIGS. 10-13 indicate that these PC-NSAID formulations can readily be aerosolized into particles having a diameter between 2-6 μm for deep lung deposition.

### Indications & Uses of Invention

We have obtained compelling preliminary evidence using a mouse model system that purified PC-NSAIDs (PC-Indomethacin and PC-Ibuprofen) administered directly to the lung or parenterally are effective in inhibiting O₃-induced pulmonary inflammation. The data from mice exposed to air that are dosed with the test agents via endotracheal administration, suggests that the PC-NSAIDs may block the acute pulmonary inflammatory response not only to pollutants and other toxicants and may also apply in the cases of smoke inhalation injury (due to fire and cigarette smoking: both primary and second-hand). Our data of the protective effect of intrapulmonary PC-NSAIDs in protecting animals from pulmonary inflammation subjected to endotracheal administration and not challenged with airborne toxicants such as O₃, also provides evidence that this invention may have utility in the treatment of lung tissue subjected to mechanical stressors, which suggests a role of this novel class of PC-NSAIDs in treating obstructive diseases of the lung such as COPD and cystic fibrosis (CF). Therefore, this novel approach describes the use of PC-associated NSAIDs, administered by a number of routes of administration, notably directly to the lung as would be delivered by aerosolization or nebulization, in addition to parenteral routes of administration to treat pulmonary inflammation in subjects with a range of pulmonary diseases including but not limited to; acute lung injury, acute respiratory distress syndrome, chronic obstructive pulmonary disease (COPD), Cystic Fibrosis, and adult respiratory distress syndrome, all of which may be exacerbated by inhalation of pollutants and allergens. This novel invention can also be used to treat acute lung injury as may occur in smoke inhalation injury or exposure to industrial/environmental toxicants such as allyl alcohol, acrolein, acrylonitrile, ammonia, arsine, chlorine, diborane, ethylene oxide, formaldehyde, hydrogen bromide, hydrogen chloride, hydrogen cyanide, hydrogen fluoride, hydrogen selenide, hydrogen sulfide, methyl hydrazine, hydrazine, methyl isocyanate, methyl mercaptan, nitrogen dioxide, nitric acid, parathion, phosgene, phosphine, sulfuric acid, sulfur dioxide, sulfur trioxide, toluene diioscyanate, or mixtures thereof. The PC-NSAIDs could be delivered as an oil or preferably as a lipidic suspension in a small volume of a biocompatible aqueous solvent (e.g. saline or PBS) either alone or in combination with a number of bronchodilators commonly used to treat these pulmonary disorders.

### Example 3

To evaluate the potential anti-inflammatory efficacy and PK/PD of a commercially available surfactant (P-SF/Curosurf™), purified (soy) PC or synthetic DPPC (dipalmitoyl-PC) in combination with ibuprofen in response to intrapulmonary administration in two murine model systems of lung injury (O₃-induced and LPS-induced pulmonary inflammation).

We used a modification the protocol employed in pilot studies. In these earlier experiments, mice were pre-dosed with phosphate buffered saline (PBS) vehicle, the test NSAID (indomethacin or ibuprofen) (at a dose of 2 mg or 5 mg NSAID/kg), PC (4 mg/k or 10 mg/kg), respectively, or the equivalent (NSAID) dose of the PC-NSAID complex using an endotracheal delivery method refined in our lab (25 μL/rat) 1 h before and 90 minutes after being exposed to either filtered room air or O₃ (2 ppm) for 3 h. Control mice were subjected to endotracheal intubation, where they received an equivalent volume of PBS before and after being exposed to room air and otherwise treated as the test animals. Both the PC-Indomethacin and PC-Ibuprofen were prepared using our proprietary method of associating the NSAID with an equimolar amount of PC (purified soy PC/Phospholipon LIPOID S100 from Lipoid, Germany or DPPC from Sigma). Purified soy PC was associated with indomethacin and synthetic DPPC (which is the prominent PC in pulmonary surfactant) was associated with ibuprofen (data which will be shown in graphic form). Twenty-four hours following the cessation of O₃ exposure, pulmonary injury and inflammation was assessed by euthanizing the animals and collecting BALF using standard techniques. The BALF was centrifuged (with the cell pellet analyzed for white cells) and the supernatant analyzed for protein, MPO activity and prostaglandin E₂, all markers of pulmonary inflammation/injury.

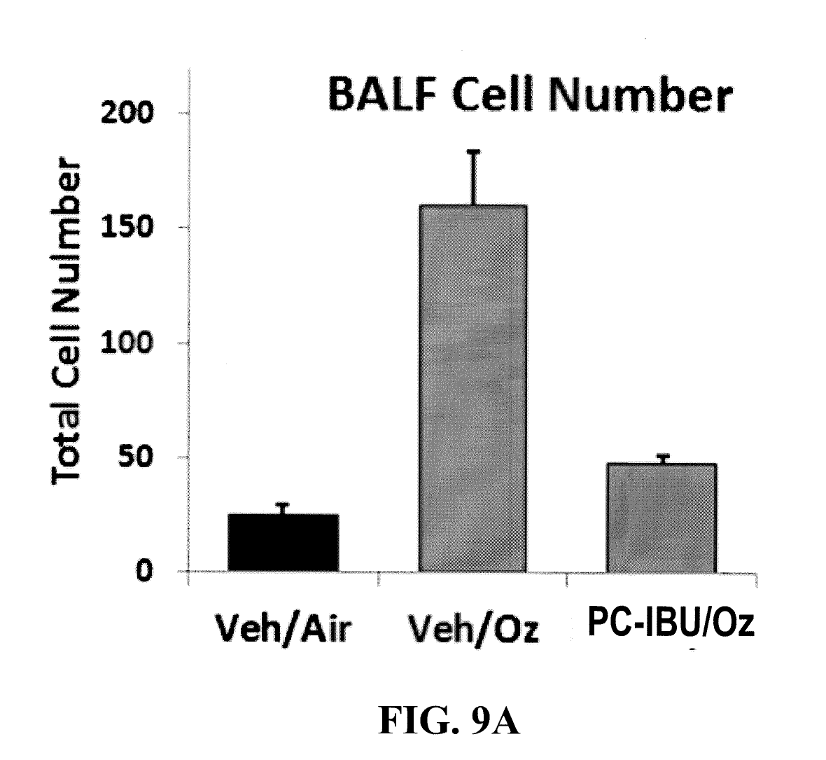

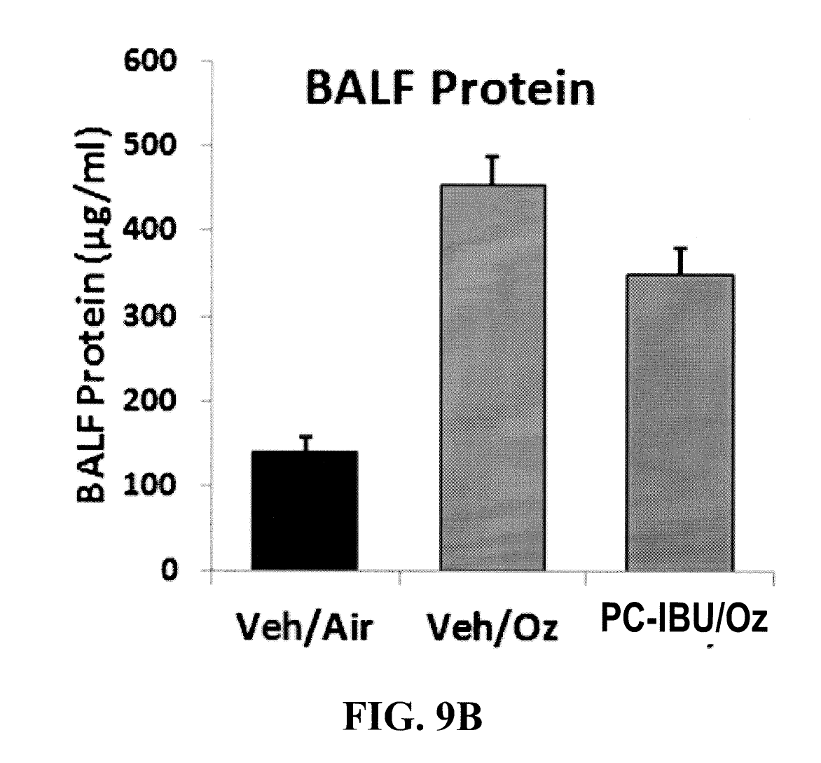

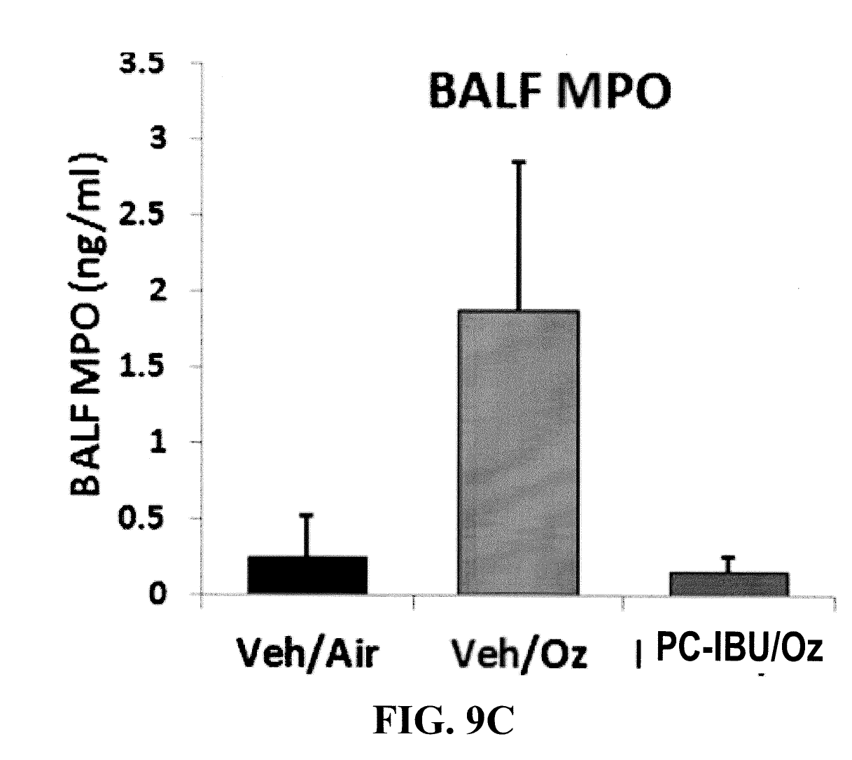

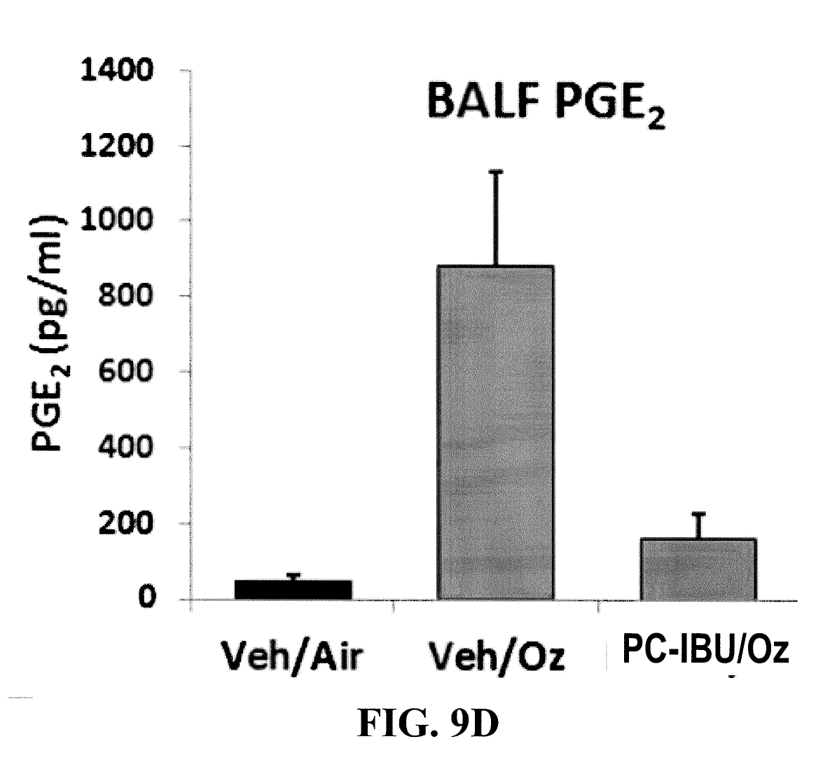

The results of these preliminary experiments are summarized below in Table 1 (for Indomethacin) and FIGS. 9A-D (for ibuprofen). In the indomethacin study, it can be seen that O3 exposure significantly (*=p<0.05 vs air) increased the white cell count, protein concentration, the neutrophil enzyme MPO and the pro-inflammatory eicosanoid PGE2, in the lavage fluid vs values of mice exposed to room air, and most importantly, endotracheal administration of PC-Indomethacin significantly reduced all of the above BALF parameters of pulmonary inflammation and barrier disruption in mice exposed to the pollutant. Interestingly, neither the NSAID nor PC on their own had a consistent effect on attenuating O₃-induced increases in cell number and the concentration of protein, MPO and PGE2 in the BALF as tabulated in Table I.

Based upon these studies, we performed a series of experiments investigating the efficacy of a combinatorial approach of administering the less toxic NSAID, ibuprofen with the natural lung surfactant, DPPC, using the O₃ model system. As can be seen in FIGS. 9A-D, ibuprofen pre-associated with DPPC (PC-Ibuprofen), utilizing our proprietary method of preparing purified PC-NSAIDs, was demonstrated to be highly efficacious in reducing BALF cells, protein, MPO activity and PGE2 concentration in mice exposed to O₃ (Oz) to values comparable to that of vehicle-treated mice exposed to room air.

TABLE I
Efficacy of PC-Indomethacin to Reverse Ozone-induced Lung Inflammation in Mice

| | | | BALF Cell | BALF Protein | BALF MPO | BALF PGE2 |
|---|---|---|---|---|---|---|
| Treatment | Ozone | N | Number | (μg/mL) | (ng/mL) | (pg/mL) |
| Vehicle | No | 19 | 79 ± 17 | 363 ± 44 | 0.10 ± 0.95 | 72 ± 30 |
| Vehicle | Yes | 17 | 153 ± 44* | 610 ± 60* | 4.26 ± 2.23* | 363 ± 86* |
| Indomethacin | Yes | 5 | 163 ± 53* | 796 ± 135* | 2.12 ± 0.43* | 274 ± 141 |
| PC | Yes | 14 | 206 ± 100 | 554 ± 47* | 2.28 ± 0.53 | 561 ± 257 |
| PC-Indomethacin | Yes | 20 | 83 ± 12 | 406 ± 40 | 0.67 ± 0.15 | 35 ± 14 |

### Example 4

### General Overview

This example relate to testing PC-NSAID compositions in preliminary aerosolization. The data was directed to PC-Ibuprofen compositions, where the PC is LIPOID S100. PC-Ibuprofen compositions were aerosolized at several different dilutions in phosphate buffered solution (PBS) using an off the shelf nebulizer.

The test system used for this study was designed to facilitate the generation, delivery and data collection of the aerosolized PC-Ibuprofen composition as lung therapeutics. The system was designed to deliver a semi-wet aerosol, which is typical for patients undergoing nebulizer drug treatment.

Particle size measurements of the aerosolized test material were measured on a calibrated (APS) TSI Model 3321 Aerodynamic Particle Sizer® (APS™) spectrometer (TSI Inc. St. Paul, Minn.). The TSI APS is a laser-diffraction particle-size system specifically designed to provide in-situ, real-time aerosol measurement data with aerodynamic particle size range between 0.5 μm and 20 μm.

Key factors for PC-Ibuprofen (PC is LIPOID S100) material that was examined in this study includes: (a) particle size distribution, (b) mass concentration, (c) mass median aerodynamic diameter (MMAD) and geometric standard deviations (GSD), and (d) estimated patient deliver rates based on particle size data for various dilution factors.

### Study Design

### System Setup

Referring now to FIG. 10{9}, a system design, generally 1000, is shown that facilitates controlled and uniform aerosol generation and delivery of the generated aerosol for testing. The system 1000 includes a using purified air tank 1002 for supplying air for aerosol generation equipped with a regulator 1004 and a metering valve 1006 and flow meter 1008 to control and monitor a flow rate of the supplied air to a nebulizer 1010. For all tests performed, the nebulizer flow rate was maintained at 8 L/min. so that the nebulizer 1010 operates in a dynamic flow through mode. The generated aerosol is then forwarded to a sealed aerosol containment plenum or test chamber 1012 for aerosol particle size distribution measurements.

The plenum 1012 is equipped with HEPA cartridge filters 1014 at an inlet 1016 and an exhaust 1018 for the introducing and exhausting purified dilution air 1020 into the plenum 1012. The purified dilution air was used to dilute and maintain a uniform and controlled flow rate of the aerosol from the plenum 1012, which is forwarded to a valve controlled exhaust system 1022 equipped with a ⅓-hp vacuum pump 1024 (Gast Manufacturing, Benton Harbor, Mich.). The system 1000 was operated at a continuous air flow of 30 L/min. for all tests performed. This provided 22 L/min. of purified dilution air in addition to the nebulizer output flow of 8 L/min. for a total system flow rate of 30 L/min. (about 1 CFM). An aerodynamic particle size (APS) sample probe 1026 was located approximately 6 inches downstream of the nebulizer 1010 and was used to measure the aerosol size distribution and concentrations in an ASP 1028. The information generated by the ASP 1028 is forwarded to a computer 1030 for data analysis and display.

### Test Sample Preparation

Two test samples were subject to aerosol characterization testing. The samples included about 4 mL of a PC-Ibuprofen (PC is LIPOID S100) composition having an Ibuprofen to PC (LIPOID S100) ratio of 2:1 PC to Ibuprofen weight ratio. The samples were stored under refrigeration until tested. Initial observation showed that the test samples were extremely viscous and would need to be diluted for aerosol characterization.

Serial dilutions of the test samples were performed using phosphate buffer saline (PBS). Serial dilutions were performed at 1:10, 1:20, 1:50, and 1:100 test sample to PBS based on mass for each formulation. Mass quantities were measured using a Mettler microbalance. Diluted test standards were prepared in sterile Falcon conical test tubes and were vigorously agitated to mix and homogenize the solutions.

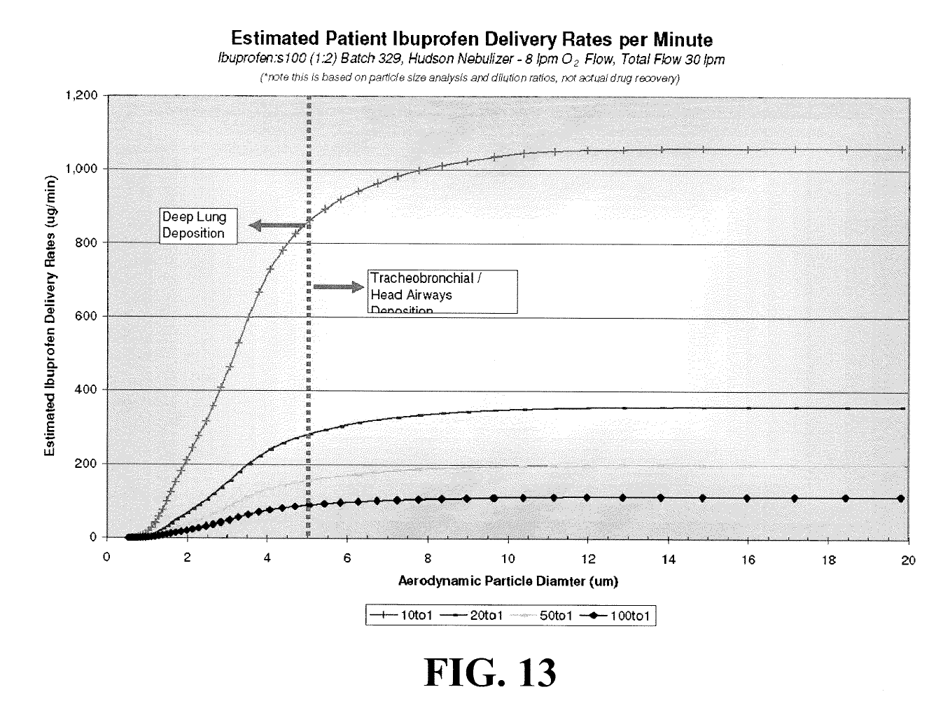

Observations for each test sample showed visible precipitation of the test samples in solution at dilutions of less than 1:100. Aerosol particle size distribution analysis testing was performed for all test sample dilutions. Mass generation of aerosol was also measured to calculate and estimate the delivered aerosol concentration by measuring the nebulizer use rate mass output in relation to total system air flow rate. It appears that the dilution should be maintained at less than about 20:1, such as about 10:1. See FIG. 13.

### Aerosol Test Samples

The aerosol test samples were prepared and runs tested as tabulated in Table II.

TABLE II
Samples Tested

| | | Dilution | Run | Nebulizer | |
|---|---|---|---|---|---|
| | | Ratio | Time | Flow | |
| Run | Material | (mass) | (min) | (lpm) | Sampling |
| 1 | PBS | — | 3 | 8 | APS |
| 2 | PBS:(PC:IBU) complex | 10:1 | 3 | 8 | APS |
| 3 | PBS:(PC:IBU) complex | 20:1 | 3 | 8 | APS |
| 4 | PBS:(PC:IBU) complex | 50:1 | 3 | 8 | APS |
| 5 | PBS:(PC:IBU) complex | 100:1 | 3 | 8 | APS |

Particle size analyses were performed based on light scattering using a TSI Aerodynamic Particle Sizer (APS) model 3321. Aerosols were generated using a Hudson RCI pneumatic nebulizer model 1895. Air used by the nebulizer was supplied by a Praxair Research Grade 5.0 O₂ tank. The nebulizer fluid used in the dilutions was MP Biomedicals, LLC—PBS Tablets cat 2810305 into filtered DI water with ibuprofen or PC-Ibuprofen (PC is LIPOID S100) at a 2:1 PC to ibuprofen weight ratio.

### Aerosol Testing Method

The method used for testing the samples for aerosol administration includes filing the nebulizer with the sample solution to be tested and positioning the nebulizer and APS sample port into test chamber, plenum. The method also includes starting the vacuum pump and adjusting the dilution flow rate. The method also includes starting dissemination with the nebulizer. The method also includes starting APS sampling and sampling continuously with APS in 20 second intervals for entire run. Finally, the nebulizer O₂ flow to the nebulizer is turned off.

### Conclusions

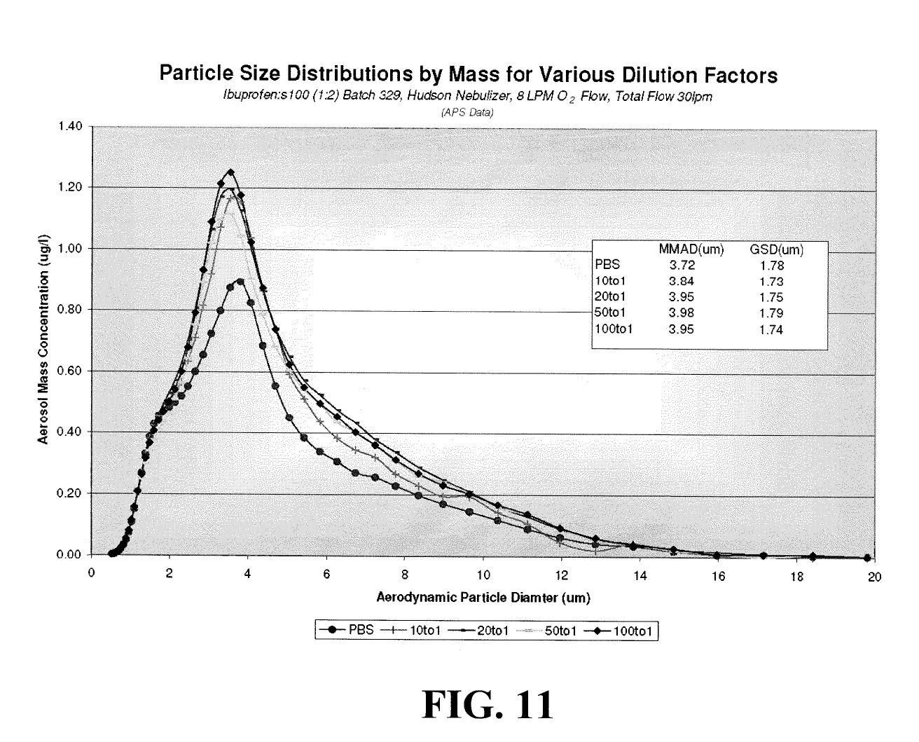

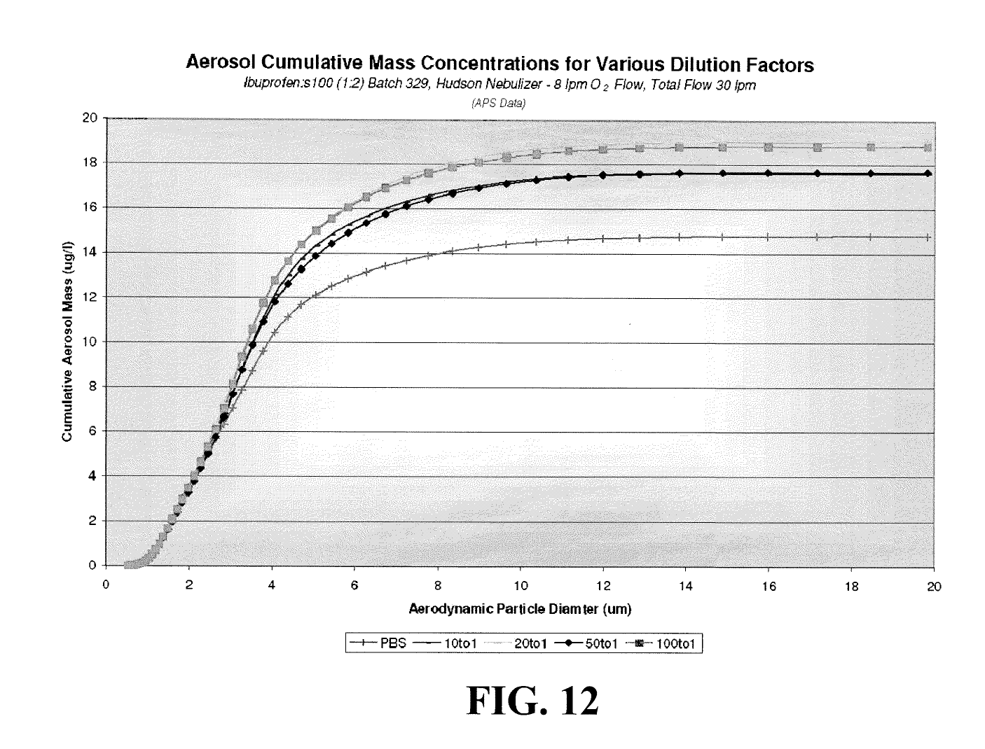

Aerosol test characterization results are shown in FIG. 11, FIG. 12 and FIG. 13. The results showed that the aerosol size distribution of the test samples at each dilution ratio in PBS have an aerosol mass median aerodynamic diameter (MMAD) of less than 4.0 μm with a geometric standard deviation (GSD) in the range of 1.75, which is comparable to the vehicle (PBS) in neat form. Please note that this is only an estimate and is based on mass concentrations as measured by the APS.

This data represents aerosol size distributions, which are near monodispersed and within the respirable mass size range for deep lung deposition, which may be effective for inhalation therapeutic delivery. The observation of precipitation of the test samples precipitating out of solution at the lower dilution ratios (small gel or particle like formation) may need further investigation for determining total solubility and accurate assessment of delivered mass of the test samples for optimizing drug delivery.

Additionally, to obtain true respirable delivery rates cascade impaction testing with analysis of the API would need to be conducted, but preliminary results suggest that pulmonary delivery in the order of 200 ug/min should be possible.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. An aerosolizable or nebulizable composition for reducing lung injury (LI), pulmonary inflammation, and/or airway hyper-responsiveness (AHR) comprising:
at least one nonsteroidal anti-inflammatory drug (NSAID), at least one zwitterionic phospholipid, and an aqueous diluent where the composition reduces lung injury (LI), pulmonary inflammation, and/or airway hyper-responsiveness (AHR), and wherein the molar ratio of the zwitterionic phospholid to the NSAID ranges from about 1:1 to about 5:1, and wherein a dilution ratio is less than about 20:1.

2. The composition of claim 1, further comprising:
at least one lung replacement surfactant composition.

3. The composition of claim 1, wherein the phospholipids are pre-associated with the NSAID.

4. The composition of claim 2, where the phospholipids are pre-associated with the NSAID and the lung replacement surfactant compositions are formulated with the NSAID.

5. The composition of claim 1, wherein the phospholipids are selected from the group of phosphatidylcholine class of phospholipids.

6. The composition of claim 1, wherein the phospholipids are selected from the group of phosphatidylcholines such as phosphatidyl choline (PC), dipalmitoylphosphatidylcholine (DPPC), other disaturated phosphatidylcholines, or mixtures and combinations thereof.

7. The composition of claim 1, wherein the lung replacement surfactant composition are selected from the group consisting of porcine lung extracts, bovine lung extracts, synthetic analogs, and mixtures or combinations thereof.

8. A method for reducing lung injury (LI), pulmonary inflammation, and/or airway hyper-responsiveness (AHR) comprising:
administering a composition comprising at least one nonsteroidal anti-inflammatory drug (NSAID) and at least one zwitterionic phospholipid, where the composition reduces lung injury (LI), pulmonary inflammation, and/or airway hyper-responsiveness (AHR).

9. The method of claim 9, further comprising:
prior to the administering step, diluting the composition with water or an aqueous carrier to form a diluted composition.

10. The method of claim 9, wherein the administering step includes:
parenterally administering the composition.

11. The method of claim 9, wherein the administering step includes:
intratracheally administering the composition.

12. The method of claim 9, wherein the intratracheally administering step includes:
intratracheally administering via inhalation.

13. The method of claim 9, wherein the intratracheally administering step includes:
intratracheally administering insufflation.

14. The method of claims 14, wherein the intratracheally administering step includes:
spraying a mist of the composition into the pulmonary system by inhalation through the throat or nose.

15. The method of claims 14, further comprising the step of:
producing the mist by atomizing the composition.

16. The method of claims 14, further comprising the step of:
producing the mist by nebulizing or aerosolizing the composition.

17. The method of claim 10, wherein the administering step includes:
parenterally administering the diluted composition.

18. The method of claim 10, wherein the administering step includes:
intratracheally administering the diluted composition.

19. The method of claim 10, wherein the intratracheally administering step includes:
intratracheally administering via inhalation.

20. The method of claim 10, wherein the intratracheally administering step includes:
intratracheally administering insufflation.

21. The method of claims 20, wherein the intratracheally administering step includes:
spraying a mist of the composition into the pulmonary system by inhalation through the throat or nose.

22. The method of claims 22, further comprising the step of:
producing the mist by atomizing the diluted composition.

23. The method of claims 22, further comprising the step of:
producing the mist by nebulizing the diluted composition.

24. The method of claims 22, wherein the mist is formed using air, oxygen, or a nitrogen and oxygen mixture.
